(19) United States
(12) Patent Application Publication
(10) Pub. No.: US 2023/0109362 A1
(43) Pub. Date: Apr. 6, 2023

(54) VIRAL BIOSENSORS

(71) Applicant: GLAXOSMITHKLINE BIOLOGICALS SA, Rixensart (BE)

(72) Inventor: Marianne DEWERCHIN, Rixensart (BE)

(73) Assignee: GLAXOSMITHKLINE BIOLOGICALS SA, Rixensart (BE)

(21) Appl. No.: 17/910,062

(22) PCT Filed: May 12, 2021
(86) PCT No.: PCT/IB2021/054065

§ 371 Date: Sep. 8, 2022

Related U.S. Application Data
(60) Provisional application No. 63/024,962, filed on May 14, 2020.

(51) Int. Cl. C12N 15/86; C07K 14/005

(52) U.S. Cl. CPC C12N 15/86; C07K 14/005; C12N 2710/10322; C12N 2710/10321; C12N 2710/10352; C12N 2830/20; C07K 2319/60; A61K 39/00

(57) ABSTRACT

The present invention relates to compositions and methods for monitoring viral synthesis inside a host cell. Compositions of the invention act as biosensors and can detect virus synthesized in a host cell in situ.

### SEQUENCE LISTING

The instant application contains a Sequence Listing which has been submitted electronically in ASCII format and is hereby incorporated by reference in its entirety. Said ASCII copy, created on Mar. 31, 2021, is named VU66850P_SL.txt and is 37,454 bytes in size.

### FIELD OF THE INVENTION

The present invention relates to the use of biosensors to monitor virus production as well as to methods of making and using the biosensors.

### BACKGROUND OF THE INVENTION

Viruses have been demonstrated to provide prophylactic and therapeutic delivery platforms whereby a nucleic acid sequence encoding a prophylactic or therapeutic molecule is incorporated into the adenoviral genome and expressed when the virus is administered to a subject. Typically, recombinant viruses are produced by a process that involves infecting mammalian cells in culture with a viral inoculum, growing the cells and harvesting the virus.

The virus enters the cell via a surface receptor, undergoes endocytosis after which the viral core is released from the endosome by acidification. The viral DNA is released and enters the nucleus, where it undergoes transcription. Following protein synthesis, viral particles reassemble and thus the viral population expands within the host cell.

Flow cytometry using a green fluorescent protein (GFP) marker has been used to demonstrate a correlation between DNA content, cell size and granularity with human adenovirus 5 and recombinant protein expression in 293 cells (Sandhu et al. (2008) Biotechnol. Prog. 24:250). Sandhu et al. expressed a GFP transgene driven by a CMV promoter independently of viral replication; the CMV promoter can drive protein expression intracellularly in the absence of viral replication, even in cells non-permissive for adenovirus. Sandhu et al. did not measure viral production in real time, e.g., in a bioreactor. Also, a fusion protein of GFP and the adenoviral protein IX has been observed to integrate into an adenoviral capsid, suggesting that protein IX can be used as a platform to anchor proteins to adenoviruses then used to follow infection and trafficking of the virus in infected cells (Meulenbroek et al. (2004) Molec. Ther. 9:617).

However, none of these approaches marks the virus itself in real time as it is synthesized. Adenoviral production processes known to date do not report infectivity or viral synthesis. Thus, there remains a need in the art for techniques that follow the viral infection and replication process and report the amount of virus as it is synthesized in a host cell.

### SUMMARY OF THE INVENTION

The invention provides a virus (a viral biosensor) that has been genetically modified to express a partial fusion. The nucleotide sequence encoding the partial fusion comprises a leaky stop codon interposed between a nucleotide sequence encoding a viral protein and a nucleotide sequence encoding a detectable marker. Expression of the partial fusion results in both an unfused viral protein and a fusion protein comprising the same viral protein coupled to a detectable marker. Thus, the viral protein and the detectable marker are expressed from a single precursor messenger RNA, with the detectable marker expressed from read-through of the leaky stop codon.

### DESCRIPTION OF THE FIGURES

FIG. 1: Diagram of adenovirus biosensors according to aspects of the invention. The triangle on the top of the icosahedral viral particle designates the capsid. Open triangles designate the absence and filled triangles designate the presence of enhanced green fluorescent protein (EGFP). Light grey circles indicate the fiber that has not fused with EGFP, open circles indicate pIX protein and dark grey circles indicate EGFP.

FIG. 2: Diagram of the sequences of adenovirus biosensors described in Examples 1-4 and in FIG. 1 (not to scale). Fragment 1 (Ad biosensor 1) shows the integration of the EGFP coding sequence (CDS) and a leaky stop codon (LS) C-terminally to the fiber coding sequence in the ChAd155 RSV genome. Fragment 2 (Ad biosensor 2) shows the integration of the EGFP coding sequence N-terminally to the fiber coding sequence. Fragment 3 (Ad biosensors 3 and 5) shows the integration of the EGFP coding sequence C-terminally to the pIX coding sequence in the ChAd155 RSV genome. Fragment 4 (Ad biosensors 4 and 6) shows the integration of an LS codon and the EGFP coding sequence C-terminally to the pIX coding sequence. The locations of the restriction sites Hpal and ASiSi are also indicated.

FIG. 3A: The DNA sequence of Fragment 1 (diagrammed in FIG. 2) (SEQ ID NO: 1). TTTAAA and GAATTC are the restriction sites DraI and EcoRI, respectively. Adenovirus is single underlined and the coding sequence of a C-terminal fragment of the fiber protein is underlined and in bold font. The leaky stop (LS) codon TGAC is in bold and italic font. CTAGGCGGGGGA (SEQ ID NO: 11) is double underlined and encodes the spacer LGGG (SEQ ID NO:12). The enhanced green fluorescent protein (EGFP) sequence is in italic font.

FIG. 3B: The fiber amino acid sequence (SEQ ID NO: 2) corresponding to Ad biosensor 1.

FIG. 3C: The amino acid sequence of the fiber protein fused to EGFP corresponding to Ad biosensor 1 (SEQ ID NO: 3). The fiber coding sequence is shown in bold font, the stop is indicated by (.), the LGGG spacer (SEQ ID NO: 12) follows the stop and the EGFP sequence is shown in italics.

FIG. 4A: The DNA sequence of Fragment 2 (diagrammed in FIG. 2) (SEQ ID NO: 4). TTTAAA and GAATTC are the restriction sites DraI and EcoRI, respectively. The coding sequence of the fiber protein is underlined. The EGFP sequence is shown in italic font.

FIG. 4B: The amino acid sequence corresponding to Ad biosensor 2 encoded by the FIG. 4A sequence (SEQ ID NO: 5).

FIG. 5A: The DNA sequence of Fragment 3 (diagrammed in FIG. 2) (SEQ ID NO: 6). TTTAAA and GAATTC represent the restriction sites Dra1 and EcoR1, respectively. Adenoviral sequence is underlined and adenoviral sequence corresponding to pIX is underlined and in bold font. The FLAG epitope is in bold font and not underlined (SEQ ID NO: 13) and the EGFP sequence is in italics.

FIG. 5B: The amino acid sequence corresponding to the Ad biosensors 3 and 5 encoded by the FIG. 5A sequence (SEQ ID NO: 7). The pIX protein coding sequence is in bold font, the FLAG epitope is DYKDDDDK (SEQ ID NO: 14) and the EGFP sequence is shown in italics.

FIG. 6A: The DNA sequence of Fragment 4 (diagrammed in FIG. 2) (SEQ ID NO: 8). TTTAAA and GAATTC are the restriction sites Dra1 and EcoR1, respectively. Adenoviral sequence is underlined and adenoviral sequence corresponding to pIX is underlined and in bold font. The TGAC leaky stop codon is in bold font and italics. The spacer is double underlined. (SEQ ID NO: 15) The EGFP coding sequence is shown in italics.

FIG. 6B: The amino acid sequence corresponding to the protein IX corresponding to Ad biosensors 4 and 6 encoded by the FIG. 6A sequence (SEQ ID NO: 9).

FIG. 6C: The amino acid sequence corresponding to Ad biosensors 4 and 6 comprising the FIG. 6A sequence (SEQ ID NO: 10). The pIX coding sequence is shown in bold font, a spacer comprising the FLAG epitope is in normal font (SEQ ID NO: 16) and the EGFP sequence is shown in italics.

FIG. 7: Western blot showing the partial fusion of EGFP with the fiber protein in Ad biosensor 1 and with the pIX protein in Ad biosensor 4 and 6. Lane L: molecular weight standards; Lanes 1, 2 and 3: Ad biosensor 1, the arrow denotes the fiber-EGFP fusion protein; Lanes 4, 5 and 6: Ad biosensor 4; Lanes 7, 8 and 9: Ad biosensor 6, the arrow denotes the pIX-EGFP fusion protein; Lane 10: ChAd155ΔE3-RSV; Lane 11: ChAd155-RSV; Lane 12: blank; Lane 13: EGFP; Lane 14: Ad biosensor 3; Lane 15: mock transfection.

FIG. 8: Fluorescence detection at 460 nm light exposure in adenoviral biosensor viral stocks following purification. Lane 1: control EGFP; Lane 3: Ad biosensor 1 fragment 1; Lane 5: Ad biosensor 4 fragment 4; Lane 7: Ad biosensor 6 fragment 6; Lane 9: ChAd155ΔE3-RSV; Lane 11: Ad biosensor 3 fragment 3. Lanes 2, 4, 6, 8, 10 and 12 are empty.

FIG. 9A: FACS analysis of the infectivity of ChAd155-RSV in a bioreactor. The solid lines show the background signal obtained with cells at 48 hours post infection and the dashed lines show the background signal at 72 hours post infection.

FIG. 9B: FACS analysis of the infectivity of Ad biosensor 4 in a bioreactor. The solid lines show the biosensor signal linked to viral infection at 48 hours and the dashed lines show the viral infection at 72 hours post infection.

FIG. 9C: FACS analysis of the infectivity of Ad biosensor 1 in a bioreactor. The solid lines show the biosensor signal linked to viral infection at 48 hours post infection and the dashed lines show the viral infection at 72 hours post infection.

### ANNOTATION OF THE SEQUENCES

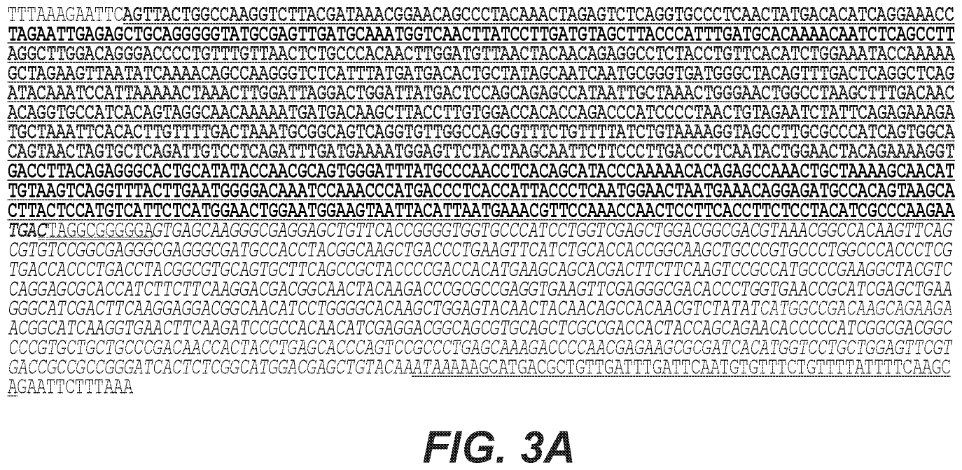

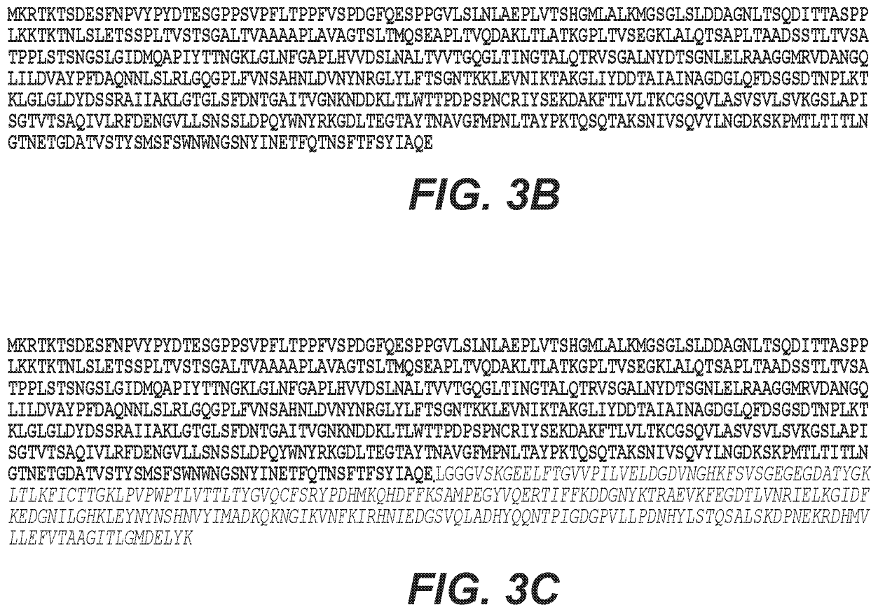

SEQ ID NO: 1—DNA sequence of fragment 1 (FIG. 3A)

SEQ ID NO: 2—Amino acid sequence of fragment 1 (fiber) (FIG. 3B)

SEQ ID NO: 3—Amino acid sequence of fragment 1 (fiber) fused to EGFP (FIG. 3C)

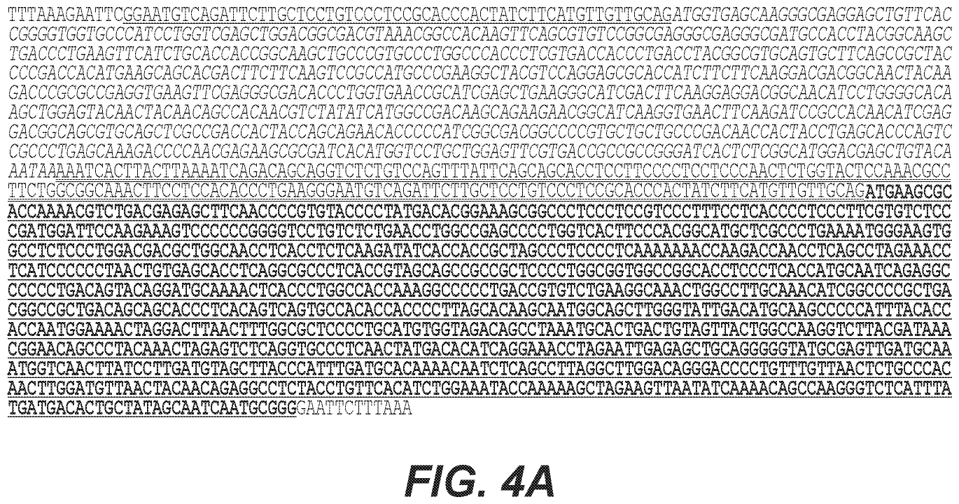

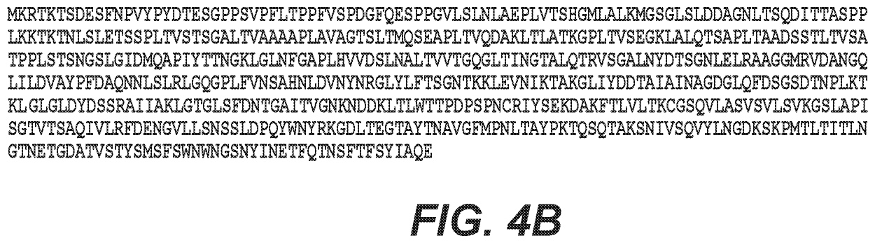

SEQ ID NO: 4—DNA sequence of fragment 2 (FIG. 4A)

SEQ ID NO: 5—Amino acid sequence of fragment 2 (FIG. 4B)

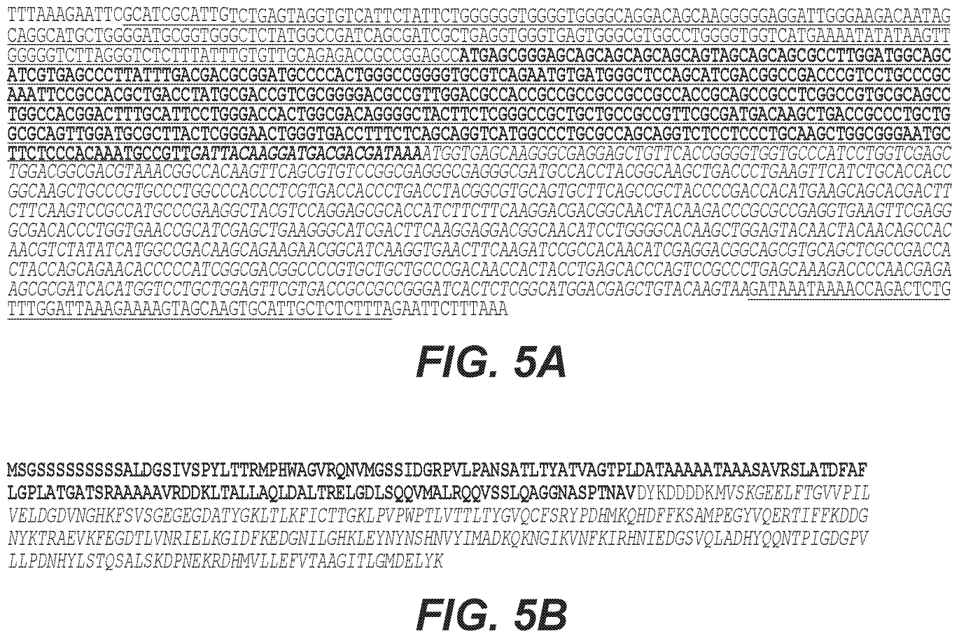

SEQ ID NO: 6—DNA sequence of fragment 3 (FIG. 5A)

SEQ ID NO: 7—Amino acid sequence of fragment 3 (pIX) (FIG. 5B)

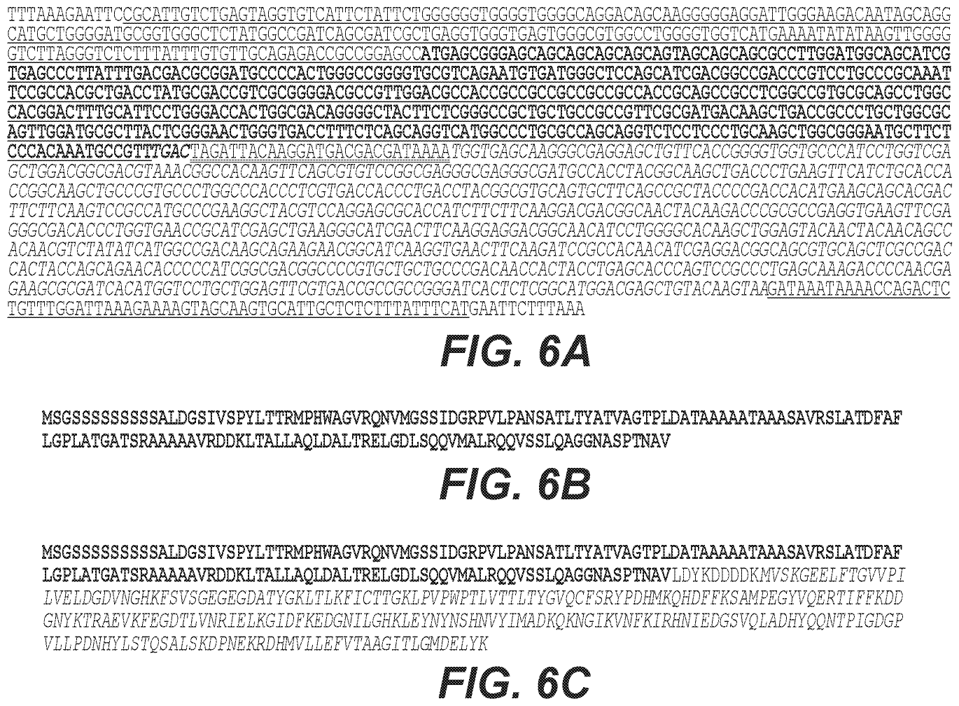

SEQ ID NO: 8—DNA sequence of fragment 4 (FIG. 6A)

SEQ ID NO: 9—Amino acid sequence of fragment 4 (pIX) (FIG. 6B)

SEQ ID NO: 10—Amino acid sequence of fragment 4 (pIX) fused to EGFP (FIG. 6C)

SEQ ID NO: 11—LGGG spacer DNA sequence

SEQ ID NO: 12—LGGG spacer amino acid sequence

SEQ ID NO: 13—FLAG epitope DNA sequence (FIG. 5A)

SEQ ID NO: 14—FLAG epitope amino acid sequence (FIG. 5B)

SEQ ID NO: 15—Spacer comprising the FLAG epitope DNA sequence (FIG. 6A)

SEQ ID NO: 16—Spacer comprising the FLAG epitope amino acid sequence (FIG. 6B)

SEQ ID NO: 17—Spacer ITRMTTIK

### DETAILED DESCRIPTION OF THE INVENTION

The invention provides viral biosensors in which a detectable marker is expressed under the control of a viral protein, i.e., to form the tagged viral protein. To achieve expression dependency, the viral genome has been modified such that the detectable marker and the viral protein are expressed from the same precursor messenger RNA and the detectable marker is expressed by read-through of a leaky stop codon. When the modified virus comprising a viral biosensor infects a cell, the marker (e.g., EGFP) is expressed during viral replication simultaneously or substantially simultaneously and in an amount proportional to the tagged viral protein. Viral biosensors of the invention provide information about the cell population that produces the virus, including viral replication and infectivity.

Viral biosensors of the invention can be constructed with any viral backbone, including adenoviruses, e.g., human and simian adenoviruses.

In a first aspect, the invention provides biosensors that report viral synthesis in real time, in situ as an infected host cell produces virus. Biosensors of the invention facilitate the measurement of the quantity of virus synthesized in the cell by reporting synthesis of a tagged viral protein. Biosensors of the invention can be tracked in situ, i.e., without removing and testing a sample during production.

“Viral biosensors” are recombinant viruses that produce partial fusions, providing both an unfused viral protein and the viral protein fused to a detectable marker. The recombinantly modified viral biosensor encodes a detectable marker and a viral protein expressed from the same precursor messenger RNA, wherein the detectable marker is expressed from read-through of a leaky stop codon. The viral biosensor emits a biosensor signal that indicates synthesis of the viral protein and is proportional to the quantity of the synthesized tagged viral protein. Recombinantly modified nucleic acids corresponding to the viral biosensors of the invention comprise a nucleotide encoding a viral protein, a leaky stop codon, e.g., a stop codon of the nucleotide encoding a viral protein of the biosensor has been replaced by a leaky stop codon, and a nucleotide encoding a detectable marker. In aspects, viral biosensors may comprise nucleotides encoding other components, such as a transgene, and may be provided as part of an expression cassette.

A partial fusion (or partial fusion protein) comprises both an unfused viral protein and a separate fusion protein comprising the same viral protein coupled to a detectable marker. The nucleotide sequence encoding the partial fusion comprises a leaky stop codon interposed between the nucleotide sequence encoding the viral protein and the nucleotide sequence encoding the detectable marker. Thus, the viral protein and the detectable marker are expressed from a single precursor messenger RNA, with the detectable marker expressed from read-through of the leaky stop codon.

A biosensor signal refers to the signal from the viral protein fused to the detectable marker (e.g., a fluorescent tag).

Real-time refers to instantaneous or substantially instantaneous monitoring of in situ viral replication or other viral processes. In aspects, real-time monitoring is achieved by a system (e.g., a microscopy system, or other system capable of measuring the detectable signal) that processes received inputs within milliseconds, allowing viral replication or other processes to be monitored within milliseconds of such process occurring.

Viral biosensors of the invention express both unfused viral protein and viral protein fused to a detectable marker (fused viral protein). The proportion of fused viral protein is quantifiable and does not cause disruption to normal viral behavior.

“Adenoviral biosensors” or “Ad biosensors” are recombinant adenoviruses that encode a detectable marker and an adenoviral protein expressed from the same precursor messenger RNA. They emit a biosensor signal that is produced by the synthesis of the adenoviral protein and is proportional to the quantity of the synthesized tagged viral protein. Adenoviral biosensors of the invention comprise a leaky stop codon, e.g., a stop codon of an adenoviral protein of the biosensor has been replaced by a leaky stop codon.

In an embodiment, the invention provides a recombinant nucleic acid encoding a fusion protein comprising a detectable marker and one or more viral proteins, wherein the detectable marker and the one or more viral proteins are expressed from the same precursor messenger RNA and wherein the nucleic acid encoding the one or more viral proteins comprises a leaky stop codon.

In an embodiment, the invention provides a recombinant nucleic acid that encodes a partial fusion protein comprising a leaky stop codon sequence interposed between a nucleotide sequence encoding a viral protein and a nucleotide sequence encoding a detectable marker; wherein the viral protein and the detectable marker are expressed from a single precursor messenger RNA and the detectable marker is expressed from read-through of the leaky stop codon; and wherein the partial fusion protein comprises the viral protein and a fusion protein comprising the viral protein coupled to the detectable marker.

In an embodiment, the invention provides a virus comprising a recombinant nucleic acid encoding a fusion protein comprising a detectable marker and one or more viral proteins, wherein the detectable marker and the one or more viral proteins are expressed from the same precursor messenger RNA and wherein the nucleic acid encoding the one or more viral proteins comprises a leaky stop codon.

In an embodiment, the human adenoviral biosensor belongs to serotype A (e.g., adenoviruses 12, 18, 31), serotype B (e.g., adenoviruses 3, 7, 11, 14, 16, 21, 34, 35, 50, 55), serotype C (e.g., adenoviruses 1, 2, 5, 6, 57), serotype D (e.g., adenoviruses 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56), serotype E (e.g., adenovirus 4), serotype F (e.g., adenoviruses 40, 41) or serotype G (e.g., adenovirus 52).

In an embodiment, the simian adenoviral biosensor is a bonobo, chimpanzee, gorilla, orangutan or rhesus macaque simian adenovirus. In an embodiment, the simian adenoviral virus is a chimpanzee virus. In an embodiment, the chimpanzee virus is AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155 (WO 2016/198621), ChAd15, SadV41, ChAd157, ChAdOx1, ChAdOx2, sAd4287, sAd4310A, sAd4312, SAdV31 or SAdV-A1337. In an embodiment, the adenovirus is a bonobo virus. In an embodiment, the bonobo virus is PanAd1, PanAd2, PanAd3, Pan 5, Pan 6, Pan 7 or Pan 9. In an embodiment, the gorilla virus comprises a vector disclosed by WO 2019/008111, e.g., GADNOU19 or GADNOU20.

In an embodiment, the biosensor is replication competent. In another embodiment, the biosensor is replication defective. A replication defective adenovirus may lack at least one gene of a genomic region selected from the group consisting of E1A, E1B, E2A, E2B, E3 and E4.

In an embodiment, an adenobiosensor comprises at least one of the following:
(a) an adenoviral 5′-end, preferably an adenoviral 5′ inverted terminal repeat;
(b) an adenoviral EIA region, or a fragment thereof selected from among the E1A_280R and E1A_243R regions;
(c) an adenoviral EIB or IX region, or a fragment thereof selected from among the group consisting of the E1B_19 K, E1B_55 K or IX regions;
(d) an adenoviral E2b region; or a fragment thereof selected from among the group consisting of the E2B_pTP, E2B_Polymerase and E2B_IVa2 regions;
(e) an adenoviral L1 region, or a fragment thereof, said fragment encoding an adenoviral protein selected from the group consisting of the L1_13.6 k protein, L1_52 k and L1_IIIa protein;
(f) an adenoviral L2 region, or a fragment thereof, said fragment encoding an adenoviral protein selected from the group consisting of the L2_penton protein according to claim 3, L2_pVII, L2_V, and L2_pX protein;
(g) an adenoviral L3 region, or a fragment thereof, said fragment encoding an adenoviral protein selected from the group consisting of the L3_pVI protein, L3_hexon protein according to claim 2 and L3_protease;
(h) an adenoviral E2A region;
(i) an adenoviral L4 region, or a fragment thereof said fragment encoding an adenoviral protein selected from the group consisting of the L4_100 k protein, the L4_33 k protein and protein L4_VIII;
(j) an adenoviral E3 region, or a fragment thereof selected from the group consisting of E3 ORF1, E3 ORF2, E3 ORF3, E3 ORF4, E3 ORF5, E3 ORF6, E3 ORF7, E3 ORF8, and E3 ORF9;
(k) an adenoviral L5 region, or a fragment thereof said fragment encoding the L5_fiber fiber protein according to claim 1;
(l) an adenoviral E4 region, or a fragment thereof selected from the group consisting of E4 ORF7, E4 ORF6, E4 ORF4, E4 ORF3, E4 ORF2, and E4 ORF1;
(m) an adenoviral VAI or VAII RNA region, preferably a heterologous adenoviral VAI or VAII RNA region, more preferably an adenoviral VAI or VAII RNA region from Ad5;
(n) an adenoviral Iva2 region;
(o) an adenoviral polymerase and/or
(p) an adenoviral 3′-end, preferably an adenoviral 3′ inverted terminal repeat.

In an embodiment, an antigen is encoded in an expression cassette comprising a transgene and regulatory elements necessary for the translation, transcription and/or expression of the transgene in a host cell. Regulatory elements may include promoters, transcription initiators, transcription terminators and enhancers. In an embodiment, a biosensor is produced by recombining a viral backbone with DNA encoding a transgene then further recombining that construct with constructs comprising a detectable marker.

In an embodiment, the transgene comprises one or more antigens. In an embodiment, the transgene encodes a polypeptide antigen. In an embodiment, the transgene encodes an RNA antigen. In an embodiment, the transgene comprises a codon optimized antigen sequence or a codon pair optimized antigen sequence. In an embodiment, the RNA antigen is a messenger RNA antigen. In an embodiment, the antigen is prophylactic or therapeutic.

In an embodiment, the detectable marker is under the regulatory control of a promoter. The promoter that regulates the detectable marker is different from the promoter that regulates the transgene.

In an embodiment, the detectable marker facilitates measurement at an early stage, an intermediate stage, or a late stage of viral synthesis.

In an embodiment, the biosensor is infectious.

In a second aspect, the invention provides a cell comprising a biosensor of the first aspect. In an embodiment, the cell is a host cell. Host cells are suitably isolated.

Host cells of the invention can be selected from any mammalian species including, without limitation, human, simian, mouse, rabbit and hamster. Non-limiting examples include 10 [symbol] /2, 3T3, A549, C2C12, fibroblast, HEK293, hepatocyte, HepG2, HT1080, L cells, myeloblast, PerC6, Procell-92, Procell-92.S, Saos, and WEHI cells. Host cells may be adherent or grown in suspension.

In a third aspect, the invention provides a method of producing a biosensor of the first aspect using a host cell of the second aspect.

### Adenoviruses

Adenoviruses are nonenveloped icosahedral viruses with a linear double stranded DNA genome of approximately 36 kb. Adenoviruses can transduce numerous cell types of several mammalian species, including both dividing and nondividing cells, without integrating into the genome of the host cell. They have been widely used for gene transfer applications due to their proven safety, ability to achieve highly efficient gene transfer in a variety of target tissues, and large transgene capacity. Human adenoviral vectors are currently used in gene therapy and vaccines.

Adenoviruses have a characteristic morphology with an icosahedral capsid comprising three major proteins, hexon (II), penton base (III) and a knobbed fiber (IV), along with a number of other minor proteins, e.g., VI, VIII, IX, IIIa and IVa2. Protein IX, among having other functions, is a molecular glue that cements the capsid proteins into place during viral assembly. It is exposed on the surface of the virion and has been reported to act as a platform for presenting autofluorescent proteins on the adenoviral surface (Parks et al. (2005) Molec. Ther. 11:19).

The fiber protein forms a trimer and each trimer protrudes from the penton base at each of the twelve vertices of the capsid. Thus, each adenovirus has 36 monomeric fiber proteins forming twelve trimers. The primary role of the fiber protein is to tether the viral capsid to the cell surface via the interaction of the knob region with a cellular receptor.

The fiber proteins of many adenovirus serotypes share a common architecture: an N-terminal tail, a central shaft made of repeating sequences, and a C-terminal globular knob domain (or “head”). The central shaft domain consists of a variable number of beta-repeats. The beta-repeats connect to form an elongated structure of three intertwined spiraling strands that is highly rigid and stable. The shaft connects the N-terminal tail with the globular knob structure, which is responsible for interaction with the target cellular receptor. The globular nature of the adenovirus knob domain presents large surfaces for binding the receptor laterally and apically. The effect of this architecture is to project the receptor-binding site far from the virus capsid, thus freeing the virus from steric constraints presented by the relatively flat capsid surface.

Although fibers of many adenovirus serotypes have the same overall architecture, they have variable amino acid sequences that influence their function as well as structure. For example, a number of exposed regions on the surface of the fiber knob present an easily adaptable receptor binding site. The globular shape of the fiber knob allows receptors to bind at the sides of the knob or on top of the fiber knob. These binding sites typically lie on surface-exposed loops connecting beta-strands that are poorly conserved among human adenoviruses. The exposed side chains on these loops give the knob a variety of surface features while preserving the tertiary and quaternary structure. For example, the electrostatic potential and charge distributions at the knob surfaces can vary due to the wide range of isoelectric points in the fiber knob sequences, varying from a pI of approximately 9 for adenovirus Ad 8, Ad 19, and Ad 37 to approximately 5 for subgroup B adenoviruses. As a structurally complex virus ligand, the fiber protein allows the presentation of a variety of binding surfaces (knob) in a number of orientations and distances (shaft) from the viral capsid.

The hexon accounts for the majority of the structural components of the capsid, which consists of 240 trimeric hexon capsomeres and 12 pentamer bases. The hexon has three conserved double barrels and the top has three towers, each tower containing a loop from each subunit that forms most of the capsid. The base of the hexon is highly conserved between adenoviral serotypes, while the surface loops are variable.

The penton is also an adenoviral capsid protein; it forms a pentameric base to which the fiber attaches. The trimeric fiber protein protrudes from the penton base at each of the twelve vertices of the capsid and is a knobbed rod-like structure. A difference in the surface of adenovirus capsids compared to that of most other icosahedral viruses is the presence of the long, thin fiber protein, whose primary role is the tethering of the viral capsid to the cell surface via its interaction with a cellular receptor.

The adenoviral genome has been well characterized. The linear, double-stranded DNA is associated with the highly basic protein VII and a small peptide pX (also termed mu). Another protein, V, is packaged with this DNA-protein complex and provides a structural link to the capsid via protein VI. There is general conservation in the overall organization of the adenoviral genome with respect to specific open reading frames being similarly positioned, e.g. the location of the early (E) E1A, E1B, E2A, E2B, E3, E4 and late (L) L1, L2, L3, L4 and L5 genes of each virus. Each extremity of the adenoviral genome comprises a sequence known as an inverted terminal repeat (ITR), which is necessary for viral replication. The 5′ end of the adenoviral genome contains the 5′ cis-elements necessary for packaging and replication; i.e., the 5′ ITR sequences (which can function as origins of replication) and the native 5′ packaging enhancer domains, which contain sequences necessary for packaging linear adenoviral genomes and enhancer elements for the E1 promoter. The 3′ end of the adenoviral genome includes 3′ cis-elements, including the ITRs, necessary for packaging and encapsidation. The virus also comprises a virus-encoded protease, which is necessary for processing some of the structural proteins required to produce infectious virions.

The structure of the adenoviral genome is described on the basis of the order in which the viral genes are expressed following host cell transduction. During adenoviral replication, different sets of genes are expressed in a cascade. Early expressed genes code for host cell control and viral genome replication, intermediately expressed genes are mostly regulatory and include pIX. Late expressed genes code for structural viral proteins, including the fiber.

In the early phase of transduction, the E1A, E1B, E2A, E2B, E3 and E4 genes of adenovirus are expressed to prepare the host cell for viral replication. The E1 gene is considered a master switch, it acts as a transcription activator and is involved in both early and late gene transcription. E2 is involved in DNA replication; E3 is involved in immune modulation and E4 regulates viral mRNA metabolism.

In an intermediate phase of transduction, protein IX and protein IVₐ₂ are transcribed intermediately. During the late phase of infection, expression of the late genes L1-L5, which encode the structural components of the viral particles, are activated.

### Adenoviral Replication

Adenoviruses can be replication competent or replication defective (WO2019/076877). Historically, adenovirus vaccine development has focused on defective, non-replicating vectors. They are rendered replication defective by deletion of the E1 region genes, which are essential for replication. Non-essential E3 and/or E4 region genes may also be deleted to make room for exogenous transgenes. An expression cassette comprising the transgene under the control of an exogenous promoter is then inserted. These replication-defective viruses are then produced in E1-complementing cells.

The term “replication-defective” or “replication-incompetent” adenovirus refers to an adenovirus that is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc., or a complete removal of a gene encoding a gene product that is essential for viral replication, such as one or more of the adenoviral genes selected from E1A, E1B, E2A, E2B, E3 and E4. Suitably, E1 and optionally E3 and/or E4 are deleted. If deleted, the aforementioned deleted gene region will suitably not be considered in the alignment when determining percent identity with respect to another sequence.

The term “replication-competent” adenovirus refers to an adenovirus which can replicate in a host cell in the absence of any recombinant helper proteins. Suitably, a “replication-competent” adenovirus comprises intact structural genes and the following intact or functionally essential early genes: E1A, E1B, E2A, E2B and E4. Wild type adenoviruses isolated from a particular animal will be replication competent in that animal.

### Transgenes

Adenoviruses may be used to deliver desired RNA or protein sequences, for example heterologous sequences, for in vivo expression. An adenovirus of the invention may include any genetic element, including DNA, RNA, a phage, transposon, cosmid, episome, plasmid or viral component. Adenoviruses of the invention may comprise an expression cassette. An “expression cassette” comprises a transgene and regulatory elements necessary for the translation, transcription and/or expression of the transgene in a host cell.

A “transgene” is a nucleic acid sequence, heterologous to the vector sequences flanking the transgene, which encodes an RNA or polypeptide of interest. The nucleic acid coding sequence is operatively linked to regulatory components in a manner which permits transgene transcription, translation, and/or expression in a host cell. In embodiments of the invention, the vectors express transgenes at a therapeutic or a prophylactic level. A “functional derivative” of a transgenic polypeptide is a modified version of a polypeptide, e.g., wherein one or more amino acids are deleted, inserted, modified or substituted.

The transgene may be used for prophylaxis or treatment, e.g., as a vaccine for inducing an immune response, to correct genetic deficiencies by correcting or replacing a defective or missing gene, or as a cancer therapeutic. As used herein, “inducing an immune response” refers to the ability of a protein to induce a T cell and/or a humoral antibody (B cell) immune response to the protein.

The composition of the transgene sequence will depend upon the use to which the resulting vector will be put. The transgene can encode a product which is useful in biology and medicine, such as a prophylactic transgene, a therapeutic transgene or an immunogenic transgene, e.g., protein or RNA. Protein transgenes and RNA include antigens. Antigenic transgenes of the invention induce an immunogenic response to a disease-causing organism. Non-limiting examples of an antigenic transgene include respiratory syncytial virus (RSV) (WO 2016/198599). RNA transgenes include tRNA, dsRNA, ribosomal RNA, catalytic RNAs, and antisense RNAs. An example of a useful RNA sequence is a sequence which extinguishes expression of a targeted nucleic acid sequence in the treated animal.

In addition to the transgene, the expression cassette also includes conventional control elements which are operably linked to the transgene in a manner that permits its transcription, translation and/or expression in a cell transfected with the adenoviral vector. As used herein, “operably linked” sequences include both expression control sequences that are contiguous with the gene of interest and expression control sequences that act in trans or at a distance to control the gene of interest.

The immune response elicited by the transgene may be an antigen specific B cell response, which produces neutralizing antibodies. The elicited immune response may be an antigen specific T cell response, which may be a systemic and/or a local response. The antigen specific T cell response may comprise a CD4+ helper T cell response, such as a response involving CD4+ T cells expressing cytokines, e.g. interferon gamma (IFN-γ), tumor necrosis factor alpha (TNF-α) and/or interleukin 2 (IL2). Alternatively, or additionally, the antigen specific T cell response comprises a CD8+ cytotoxic T cell response, such as a response involving CD8+ T cells expressing cytokines, e.g., IFN-γ, TNF-α and/or IL2.

An “immunologically effective amount” is the amount of an active component sufficient to elicit either an antibody or a T cell response or both sufficient to have a beneficial effect, e.g., a prophylactic or therapeutic effect, on the subject.

The expression cassette comprising the transgene may also include a reporter sequence, which upon expression produces a detectable biosensor signal. Such reporter sequences include, without limitation, DNA sequences encoding beta-lactamase, beta-galactosidase (LacZ), alkaline phosphatase, thymidine kinase, green fluorescent protein (GFP), chloramphenicol acetyltransferase (CAT), luciferase, membrane bound proteins including, for example, CD2+, CD4+, CD8+, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting (FACS) assays and immunological assays, including enzyme linked immunosorbent assay (ELISA), radioimmunoassay (RIA) and immunohistochemistry. The reporter sequence is different from the detectable marker of the biosensor.

A transgene of the invention or an immunogenic derivative or fragment thereof may comprise a codon optimized nucleic acid sequence. By “codon optimized” it is meant that modifications in the codon composition of a recombinant nucleic acid are made without altering the amino acid sequence. Codon optimization has been used to improve mRNA expression in different organisms by using organism-specific codon-usage frequencies.

A transgene of the invention or an immunogenic derivative or fragment thereof may comprise a codon pair optimized nucleic acid sequence. In addition to, and independently from, codon bias some synonymous codon pairs are used more frequently than others. This codon pair bias means that some codon pairs are overrepresented and others are underrepresented. By “codon pair optimized,” it is meant that modifications in the codon pairing are made without altering the amino acid sequence.

### Sequence Identity

Identity with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 or swgapdnamt can be used in conjunction with the computer program. In an embodiment, the gap opening penalty is 15, the gap extension penalty is 6.66, the gap separation penalty range is eight and the percent identity for alignment delay is 40. By way of example, the percent identity can be calculated as the total number of identical matches multiplied by 100 and then divided by the sum of the length of the longer sequence within the matched span and the number of gaps introduced into the shorter sequences in order to align the two sequences. Where the present disclosure refers to a sequence by reference to a UniProt or GenBank accession code, the sequence referred to is the current version as of the filing date of the present application.

The skilled person will recognize that individual substitutions, deletions or additions to a protein which alters, adds or deletes a single amino acid or a small percentage of amino acids is an “immunogenic derivative” where the alteration(s) results in the substitution of an amino acid with a functionally similar amino acid or the substitution/deletion/addition of residues which do not impact the immunogenic function.

### Viral Biosensors

The availability of large quantities of adenovirus is necessary to conduct studies and produce bioactive commercial grade material. An efficient, scalable and reproducible process for producing adenovirus maximizes the viral yield on a per cell and/or a per volume basis while also maintaining bioactivity. Viral yield can be measured by counting viral particles and viral bioactivity can be measured as the infectivity of the virus following infection of a permissive cell line.

Biomarkers have been used to monitor the production of virus and recombinant protein. Monica et al. tested methods for monitoring infection, including metabolic rate analysis, respiration, cell size, cell number, cell viability and changes in capacitance (Monica et al. (2000) Biotechnol. Prog. 16:866). They noted that, while providing a positive indication of infection, the biomarkers were not always specific for infection; also, the need for repeated sampling limited their utility in a manufacturing process. Henry et al. measured infection status by monitoring respiration, fluorescence and biovolume, sampling at time points following infection (Henry et al. (2004) Biotechnol. Bioeng. 86:765). Sandhu et al. used flow cytometry to measure cell size, granularity and DNA content and to correlate these parameters with the production of the human adenovirus Ad5 (Sandhu et al. (2008) Biotechnol. Prog. 24:250). Meulenbroek et al. fused green fluorescent protein (GFP) to adenoviral pIX, incorporated the fusion protein into the viral capsid and followed the virus by fluorescent microscopy (Meulenbroek et al. (2008) Molecular Therapy 9:617). However, none of these biomarkers measure viral production and infectivity in real time during viral production.

Adenoviral biosensors of the invention follow adenoviral protein synthesis in real time, as synthesis is occurring inside the host cell. They provide a readout that indicates a viral stage and is proportional to the quantity of labeled/tagged virus synthesized per cell in a bioreactor. The biosensors express a detectable marker/tag under the same regulatory control element as a selected viral protein, i.e., the tagged viral protein. To obtain this expression dependency, the adenoviral genome is modified to express the detectable marker and the viral protein from the same precursor messenger RNA and the detectable marker is expressed from read-through of a leaky stop codon to produce the tagged viral protein. Thus, a single precursor messenger RNA species can result in expression of the viral protein alone as well as the viral protein fused to the detectable marker. “Precursor messenger RNA” is an RNA transcript that is further processed to messenger RNA. When the modified adenovirus infects a host cell, the tag is expressed simultaneously and proportionally to the tagged viral protein. The amount of the expressed tag can be measured by any known means and the readout informs the efficiency of viral replication.

In an embodiment, biosensors of the invention enable convenient monitoring of adenoviral protein synthesis and therefore infection and viral particle synthesis. They produce a biosensor signal that is proportional to the quantity of the tagged viral protein synthesized because the virus itself carries the marker. Biosensors of the invention allow the monitoring of both infection and protein synthesis in real time, on a per cell basis. The adenoviral genome is modified by introducing a detectable marker gene such that the expression of the marker depends directly upon the expression of an adenoviral protein. The biosensor signal emitted by the detectable marker is proportional to the amount of incorporated marker gene and expression thereof and thus proportional to the amount of tagged viral protein.

“Detectable marker genes” encode an RNA or a protein that can be identified by a laboratory test. Detectable marker genes of the invention can be any known in the art. Examples of detectable marker genes include genes that encode affinity tags, epitope tags and fluorescent tags. Affinity tagged proteins include but are not limited to glutathione-S transferase, poly-histidine, calmodulin binding protein and maltose binding protein. Epitope tags include but are not limited to myc tags, human influenza hemagglutinin and FLAG.

Fluorescent tags, i.e., fluorescein fluorophores are known in the art and commercially available. They include but are not limited to red fluorescent protein and green fluorescent protein (GFP), which is isolated from the jellyfish Aequorea Victoria. GFP emits a bright green fluorescence that does not fade easily when exposed to blue or ultraviolet light. Fluorescent tags include fluorescein derivatives, e.g., enhanced GFP (EGFP), and GFP that has been mutated so that the fluorescence spectrum shifts, e.g., to blue, cyan or yellow variants. Fluorescent tags may be rhodamine or eosin derivatives. Fluorescent tags also include but are not limited to mTagBFP2, mTurquoise2, mCerulean3, EGFP, mWasabi, superfolder GFP, mNeonGreen, mClover3, Venus, Citrine, tdTomato and TagRFP-T.

Other genetically encoded tagging strategies include SNAP tag, SNAP_{f} tag, CLIP tag, CLIP_{f} tag, Halo tag, TMP tag, Sun tag, GFP1-10/GFP11, sfCherryl-10/sfCherry11, RNA tags, F30-Broccoli, Mango, DNB aptamer and JX1 aptamer.

In an embodiment, a protein spacer is included in the biosensor. In an embodiment, the protein spacer is ITRMTTIK (SEQ ID NO: 17). In an embodiment, the protein spacer is the FLAG sequence (SEQ ID NOS: 13 and 14). In an embodiment, the protein spacer comprises the FLAG sequence (SEQ ID NO: 15). The FLAG sequence can act as an antigenic epitope, a spacer or both.

The expression of the tagged viral protein encoded by the biosensor is distinct from the expression of a transgene encoded by the biosensor. The transgenes are regulated by a transgene-specific promoter and are translated separately from adenoviral proteins. The transgene may be translated in the host cell whether or not the adenovirus is replicating in the host cell. The quantity of the expressed detectable marker can be measured by fluorescent activated cell sorting (FACS), fluorescent microscopy or any known detection method.

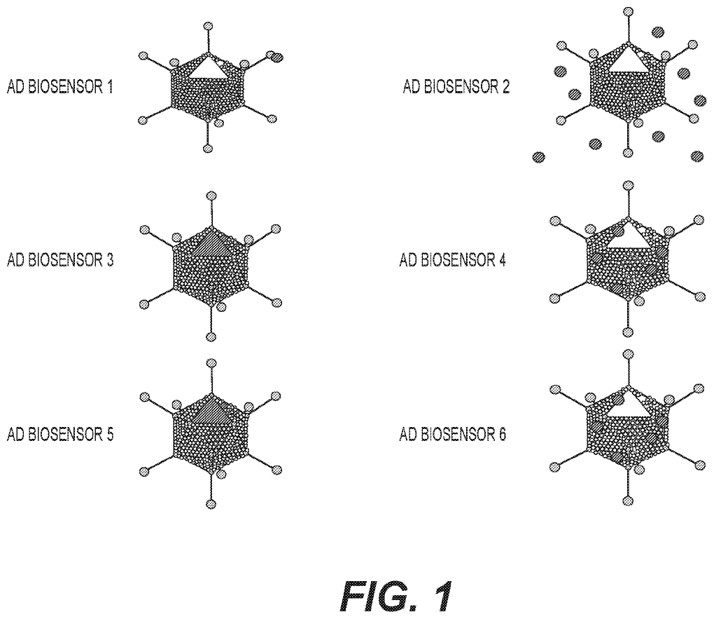

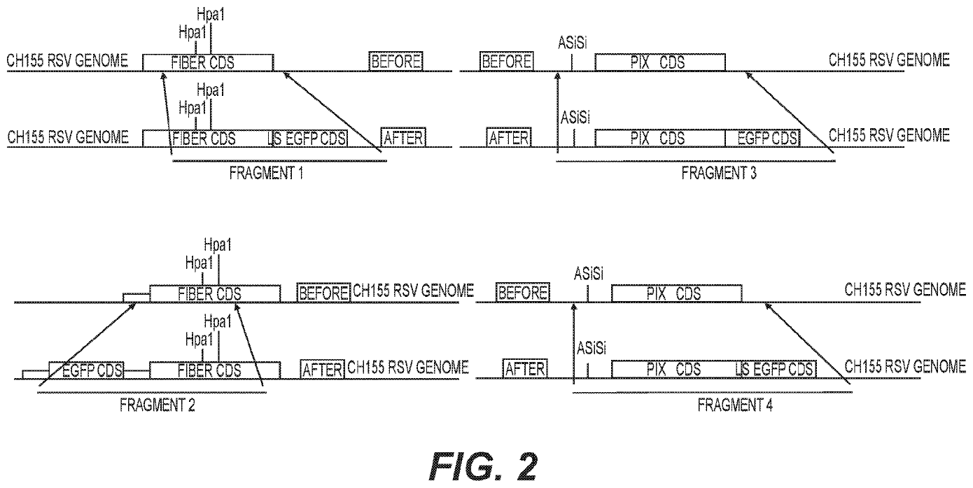

Ad biosensor 1 (FIG. 1 and FIG. 2 (Fragment 1)) expresses a partial fusion of an adenovirus fiber protein with enhanced green fluorescent protein (EGFP). The E3 region of the adenovirus is deleted. A leaky stop (LS) codon with the sequence TGAC replaces the natural stop codon of the fiber protein, allowing EGFP to fuse to the fiber protein at the fiber C-terminus when, and only when the stop codon is skipped. At the LS, the codon at the junction between the adenoviral protein and the detectable marker was replaced with a sequence coding for the amino acid sequence LGGG (SEQ ID NO: 12) to provide steric flexibility between the fiber and the EGFP detectable marker. The EGFP codon next to the stop codon was changed from AAG to AAA to restore the 5′ polyA signal.

As a result of the design, only a fraction of the fibers that are produced are fused to EGFP. The proportion is high enough to be easily quantified but low enough so as not to cause disruption to normal viral behavior. For example, the proportion of fusion can be varied to range from about 0.1% to about 10%, optionally about 1% to about 8%, about 2% to about 6%, or about 5%, depending on the choice of host cell and the amount of LS tRNA. As described above, fibers form trimers in the assembled viral capsid and the tagged fiber monomers trimerize with untagged fibers.

The fiber protein is expressed late in viral protein synthesis, thus adeno biosensor 1 indicates late viral synthesis. A fluorescent signal indicates the expression of the fiber protein and has a leaky stop codon that limits the amount of viral protein that fuses with the fluorescent marker. Ad biosensor 1 allows tracking of the path of the virus as it is assembled by the host cell, beginning from the step of late protein synthesis.

In an embodiment, Ad biosensor 1 comprises a simian adenoviral backbone. In a particular embodiment, the backbone is a chimpanzee adenoviral backbone. In a more particular embodiment, the chimpanzee adenoviral backbone is ChAd155. In an embodiment, Ad biosensor 1 encodes a transgene. In an embodiment, the transgene is antigenic.

Ad biosensor 2 (FIG. 1 and FIG. 2 (Fragment 2)) expresses the detectable marker EGFP simultaneously with the adenoviral fiber protein. The E3 region of the adenovirus is deleted. A splice acceptor was added to the EGFP gene as part of a 902 bp fragment composed of the splice acceptor, the 150 bp upstream of the fiber ATG initiation codon and the GFP coding sequence. EGFP is expressed freely and simultaneously with the viral fiber from the same precursor mRNA.

As a result of this design, the fiber and the EGFP tag are not fused. The signal emitted by Ad biosensor 2 correlates with late viral protein synthesis however the viral particles themselves are not fluorescent.

Ad biosensor 3 (FIG. 1 and FIG. 2 (Fragment 3)) expresses a fusion protein of pIX and EGFP. The E3 region of the adenovirus is deleted. The EGFP marker is expressed in fusion with the pIX protein at a 100% fusion efficiency; i.e., every pIX protein is tagged with the detectable marker and all viral particles are highly fluorescent. The junction between pIX and EGFP is modified with a short sequence encoding the FLAG sequence, which allows detection by western blot and provides steric flexibility between the two proteins.

As a result of this design, the fusion protein replicated in the genome but the infectivity of the resulting viruses was below the level of detection. Protein IX is expressed during the intermediate phase of viral protein synthesis, thus Ad biosensor 3 is expressed during intermediate viral synthesis.

Ad biosensor 4 (FIG. 1 and FIG. 2 (Fragment 4)) expresses a partial fusion of an adenovirus pIX protein with EGFP. The E3 region of the adenovirus is deleted. A leaky stop (LS) codon with the sequence TGAC replaces the natural stop codon of the pIX protein, allowing EGFP to fuse with the pIX protein at its C-terminus when, and only when, the stop codon is skipped. At the LS, the naturally occurring junction was replaced with a sequence encoding a spacer encoding the FLAG sequence, which allows detection by western blotting and provides steric flexibility between the partially fused proteins.

As a result of this design, only a fraction of the pIX fuses to EGFP. The ratio of fusion can be varied to range from about 0.1% to about 10%, depending on the choice of host cell and the amount of LS tRNA. All or most of the assembled viral particles are fluorescent.

The pIX protein is expressed at an intermediate stage of viral protein synthesis, thus Ad biosensor 4 indicates intermediate viral synthesis. A fluorescent signal is produced with the expression of the pIX protein and the leaky stop codon limits the amount of viral protein that fuses with the fluorescent marker. Ad biosensor 4 tracks the path of the virus as it is assembled in the host cell, beginning from the step of intermediate viral protein synthesis.

In an embodiment, Ad biosensor 4 comprises a simian adenoviral backbone. In a particular embodiment, the backbone is a chimpanzee adenoviral backbone. In a more particular embodiment, the chimpanzee adenoviral backbone is ChAd155. In an even more particular embodiment, the ChAd155 backbone has the E3 region deleted. In an embodiment, Ad biosensor 4 encodes a transgene. In an embodiment, the transgene is antigenic.

Ad biosensor 5 (FIG. 1) is similar to Ad biosensor 3, however the E3 region has not been deleted. Ad biosensor 6 (FIG. 1) is similar to Ad biosensor 4, however the E3 region has not been deleted.

Biosensors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

The biosensors provided herein allow for in situ monitoring of viral processes, e.g., in a bioreactor without obtaining a sample of the reaction. The biosensors allow detection of viral processes such as synthesis at particular stages (early, intermediate, or late) without disrupting normal viral function. Additionally, the biosensors may comprise a transgene comprising a therapeutic or prophylactic. In aspects, this may allow manufacturing of the transgene to be improved by monitoring of particular viral stages in the bioreactor.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” in relation to a numerical value is optional and means, e.g., the amount ±10%.

The term “comprising” encompasses “including” as well as “consisting,” e.g., a composition comprising X may consist exclusively of X or may include something additional, e.g., X+Y. The term “substantially” does not exclude “completely.” For example, a composition that is substantially free from Z may be completely free from Z.

The following examples are provided to illustrate certain particular features and/or embodiments. These examples should not be construed to limit the invention to the particular features or embodiments described.

### EXAMPLES

### Example 1: Adenovirus Biosensor Construction

Six types of adenoviral biosensors were constructed to test the effect of different marker conditions and monitor different steps of viral protein synthesis. The adenoviral proteins fiber and pIX were selected as examples of biosensor tagged proteins because they are naturally expressed from different promoters and at different time points of the infection cycle; pIX is expressed at an intermediate stage of adenoviral protein synthesis and the fiber capsid protein is expressed at a late stage. Also, pIX and fiber exhibit different expression levels; the fiber is more abundantly expressed and this difference allows measurement across a broad dynamic range.

Adenoviral genomes have a limit to the size of additional DNA they can accommodate while maintaining their ability to properly assemble. To limit any theoretical misassembly due to insertion of a detectable marker that increases the genome size, in some of the Ad biosensors the E3 region was deleted (ΔE3) from the adenovirus to shorten the genome, allowing more room for the inserted sequences. The E3 region is dispensable for viral replication and its deletion is known not to impact productivity. Ad biosensors 1, 2, 3, and 4 were introduced into an adenoviral backbone with an E3 deletion (n). For comparison, Ad biosensors 5 and 6 were constructed with an intact E3 region and found to be comparable to Ad biosensors 3 and 4, respectively, as shown in the examples below.

ChAd155-RSV was prepared as previously described (WO 2016198599) and the E3 region deleted according to known procedures. Recombinant adenoviral genomes were constructed by recombining the ChAd155 or ChAd155 ΔE3 backbone with DNA encoding respiratory syncytial virus (RSV) transgenes then recombining the ChAd155-RSV or ChAd155 ΔE3-RSV with DNA encoding the constructs shown in FIGS. 3A, 4A, 5A and 6A to produce Ad biosensors 1, 2, 3 and 4 respectively as shown in Table 1 (see also, FIG. 2).

TABLE 1

| Ad | Adenoviral | Antigenic | Tagged | | | Leaky Stop |
|---|---|---|---|---|---|---|
| Biosensor | Backbone | Transgene | Protein | Marker | Fusion | Codon |
| 1 | ChAd155ΔE3 | RSV | Fiber | EGFP | Partial fusion | yes |
| 2 | ChAd155ΔE3 | RSV | Fiber (not | EGFP | None (free EGFP) | no |
| | | | fused) | | | |
| 3 | ChAd155ΔE3 | RSV | pIX | EGFP | Fusion | no |
| 4 | ChAd155ΔE3 | RSV | pIX | EGFP | Partial fusion | yes |
| 5 | ChAd155 | RSV | pIX | EGFP | Fusion | no |
| 6 | ChAd155 | RSV | pIX | EGFP | Partial fusion | yes |

Linearized plasmids and the corresponding fragments were mixed and transformed in E. coli BJ5183, resulting in the recombinant genomes. The recombinant clones were selected by PCR, plasmid DNA was prepared and sequenced using the Sanger method, the sequences were confirmed and subsequent restriction analysis confirmed the integrity of the Ad biosensors. Plasmid DNAs were linearized by digestion with Pme1 then transfected with Lipofectamine 2000 cationic lipid into Procell-92.S host cells for rescue. After twelve days of transfection, the rescued viruses were harvested and used to inoculate fresh cells and grown in DMEM with 10% fetal bovine serum and 6 mM glutamine. Fluorescence was monitored during the transfection by microscopy. All six Ad biosensors fluoresced in these host cells, demonstrating that the constructs were expressed by the host cells. Viral stocks of all six Ad biosensors were obtained following the transfection of the host cells, demonstrating that the resulting Ad biosensor viral particles were infectious.

### Example 2: Adenovirus Biosensor Characterization

The adenovirus biosensors were plaque purified and the clones amplified until a full cytopathic effect (CPE) was observed with Ad biosensors 1, 3 and 4. Biosensor 2 clones did not attain a full CPE and microscopic examination revealed that while only a small percentage of cells displayed the fluorescent detectable marker, their fluorescence intensity was very high. This indicated that few infectious adenoviruses were properly assembled and, accordingly, Ad biosensor 2 was not further characterized.

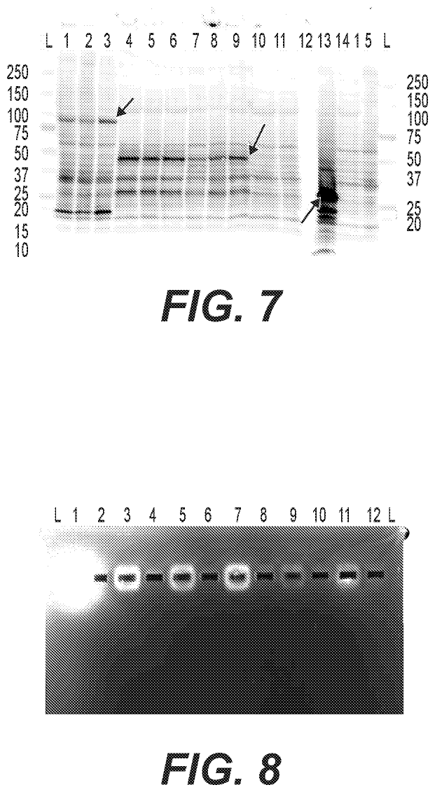

Cultures infected with Ad biosensors 1, 3, 4, 5 and 6 were harvested and the fusion proteins were detected by western blot. FIG. 7 shows western blot evidence that EGFP has fused to the fiber and to pIX in Ad biosensors 1, 4 and 6. Host cells permissive for adenoviral infection were infected for three days, the cells were harvested and fifty micrograms of total cellular protein were loaded into each lane of a reducing SDS-PAGE gel and transferred to a membrane. The membrane was incubated with a rabbit anti-EGFP antibody (Rockland 600-401-215) at a dilution of 1:10,000 followed by a horseradish peroxidase (HRP) labeled polyclonal goat anti-rabbit antibody (Dako P0448) at a dilution of 1:5000. HRP was then detected using the Amersham ECL Prime Western Blotting Detection Reagent (RPN2236).

Lanes L contain the molecular weight markers Ladder BioRad Precision Plus All Blue. Lanes 1-3: Ad biosensor 1; the arrow at lane 3 denotes the fiber-EGFP fusion protein in Ad biosensor 1. Lanes 4-6: Ad biosensor 4; lanes 7-9: Ad biosensor 6, the arrow at lane 9 denotes the pIX-EGFP fusion protein in Ad biosensors 4 and 6. Lane 10: ChAd155ΔE3-RSV; lane 11: ChAd155-RSV; lane 12: blank; lane 13: EGFP; Lane 14: Ad biosensor 3; Lane 15: mock transfection.

The fiber protein, having a molecular weight of approximately 61 kD, was detected in all of the tested adenoviruses. The clones expressing Ad biosensor 1 showed the increased molecular weight pattern of approximately 90 kDa expected of an EGFP fusion with the fiber (the molecular weight of EGFP is approximately 30 kDa).

Also shown in FIG. 7, the pIX protein having a molecular weight of approximately 15 kDa was also detected in all the tested adenoviruses, although it is present in lower amounts than the fiber. The clones expressing Ad biosensors 4 and 6 showed the increased molecular weight pattern of approximately 45 kDa expected of an EGFP fusion with pIX.

Additional western blots with anti-penton and anti-GFP antibodies demonstrated that the cultures from Ad biosensor 1, Ad biosensor 4 and Ad biosensor 6 demonstrated protein patterns like those of ChAd155-RSV, i.e., the expression level of the fibers and pentons were comparable to those of ChAd155-RSV native virus. The blots of the cultures infected with Ad biosensor 3 and Ad biosensor 5 showed a low level of fiber and penton expression, indicating that viral assembly was inefficient.

To further determine whether the GFP marker was incorporated into the Ad biosensors such that the viral particles themselves were fluorescent, Ad biosensor 1, 3, 4 and 6 were purified using a Biomiga kit, the supernatants loaded onto a column, then washed and eluted. Fifty microliters of each eluate were then loaded onto alternating lanes of a 1% agarose gel, as shown in FIG. 8. Strong fluorescence was observed with the positive control EGFP amplien TF25 (N68 846-38) and with Ad biosensor fragments 1, 4 and 6 and to a lesser extent with biosensor 3, confirming the efficient incorporation of the detectable marker into Ad biosensors 1, 4 and 6.

### Example 3: Adenovirus Biosensor Productivity and Infectivity

The productivity and infectivity of biosensors 1, 3, 4 and 6 were evaluated using qPCR, HPLC, and an infectivity assay. PCR was performed according to known methods. The infectious virus concentration was determined by titration on human embryonic kidney (HEK293) cells and reported as infectious units per milliliter (IU/ml). Microtiter plates containing HEK293 cells were inoculated with serial dilutions of the test samples and incubated for 24 hours at 37° C.±1° C., with the dilution medium as a negative control. Samples from Ad biosensors 1 and 3 were assayed in triplicate. A monovalent virus preparation of homologous strain and of known titer was used as an internal control. Following incubation, the potency was evaluated by immunofluorescence with a flow cytometer. IU/ml was calculated using a Poisson's law-based formula and the results are shown in Table 2 below.

TABLE 2

| | qPCR titer | IU titer | Infectivity Ratio |
|---|---|---|---|
| Construct | (vp/ml) | (IU/ml) | (qPCR/IU) |
| ChAd155-RSV ΔE3 | 4.63 × 10¹⁰ | 4.97 × 10⁸ | 93 |
| ChAd155-RSV | 3.88 × 10¹⁰ | 4.24 × 10⁸ | 92 |
| Ad biosensor 1 | 8.13 × 10⁹ | 8.21 × 10⁷ | 99 |
| | 9.09 × 10⁹ | 8.28 × 10⁷ | 110 |
| | 7.83 × 10⁹ | 8.65 × 10⁷ | 91 |
| Ad biosensor 3 | 2.16 × 10¹⁰ | undetectable | undetectable |
| Ad biosensor 4 | 3.79 × 10¹⁰ | 4.15 × 10⁸ | 91 |
| | 3.36 × 10¹⁰ | 4.09 × 10⁸ | 82 |
| | 3.35 × 10¹⁰ | 4.44 × 10⁸ | 75 |
| Ad biosensor 6 | 2.14 × 10¹⁰ | 2.42 × 10⁸ | 88 |
| | 2.74 × 10¹⁰ | 3.13 × 10⁸ | 88 |
| | 3.46 × 10¹⁰ | 3.34 × 10⁸ | 104 |

The Ad biosensors 1, 4 and 6 were comparably productive to the native adenovirus when cultured under the same conditions. These results indicate that Ad biosensors 1, 4 and 6 can replicate, assemble and infect cells in a manner comparable to the parent adenovirus. Ad biosensor 3 was not able to efficiently infect the host cells, as its infectivity was below the level of detection by the assay.

### Example 4: Adenovirus Biosensor Productivity and Infectivity in an Industrial Bioreactor

Ad biosensors 1 and 4 were produced in ProCell-92.S host cells grown in suspension in CD293 media with 6 mM glutamine and 5 mg/I insulin. These Ad biosensors then infected Procell-92.S host cells in separate bioreactors, in parallel. The cells were infected with Ad biosensor 1 at a multiplicity of infection (MOI) of 50 viral particles per cell and a cell concentration of 0.5×10⁶ cells/ml. The cells were infected with Ad biosensor 4 or, as a control, adenovirus ChAd155-RSV at a multiplicity of infection (MOI) of 50 viral particles per cell and a cell concentration of 1.5×10⁶ cells/ml.

Cells infected with Ad biosensors 1 and 4 were harvested at 0, 6 h, 24 h, 30 h, 48 h, 54 h and 72 h after infection and assayed by FACS analysis. Cells infected with control ChAd155-RSV were sampled at 48 h and 72 h. Samples of control virus, Ad biosensor 1 and Ad biosensor 4 taken at 48 h and 72 h were analyzed for phenotype and GFP expression without further processing; samples taken at 0, 6 h, 24 h, 30 h, 48 h, 54 h and 72 h were frozen; they tested comparably to the unfrozen samples.

Following cell harvest, viral production and the infectivity of the viruses produced in the bioreactor were evaluated by qPCR, HPLC and an infectivity test as described above. Ad biosensors 1 and 4 were compared to data obtained with ChAd155 under the same culture and infection processes. The results are shown in Table 3 below.

TABLE 3

| | | | | Infectivity | Specific |
|---|---|---|---|---|---|
| | qPCR | HPLC | Viral titre | ratio | productivity |
| Construct | vp/ml | PU/ml | IU/ml | IU/vp | PU/cell |
| ChAd155-RSV | 1.11 × 10¹¹ | 1.20 × 10¹¹ | n/d | 50-150 | 80,000 |
| Ad biosensor 1 | 1.80 × 10¹⁰ | 1.49 × 10¹⁰ | 1.71 × 10⁸ | 105 | 30,000 |
| Ad biosensor 4 | 1.67 × 10¹¹ | 1.05 × 10¹¹ | 1.64 × 10⁹ | 102 | 70,000 |

In the bioreactor, Ad biosensor 4 was as productive as the control adenovirus, measured as viral particle units per cell. Biosensor 1 was less productive; however, both Ad biosensors were able to infect the host cells with the same efficiency as the control virus.

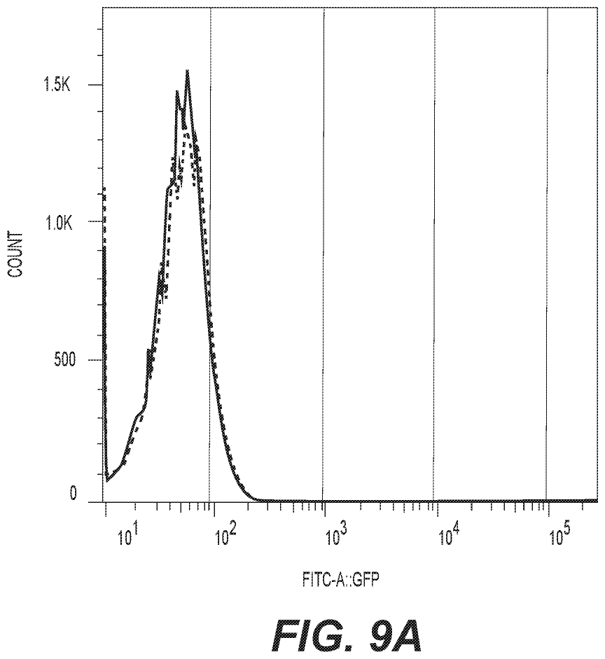

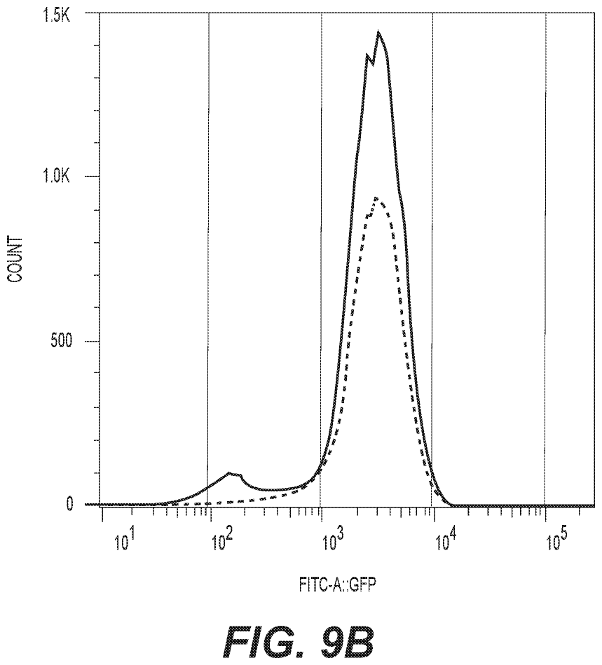

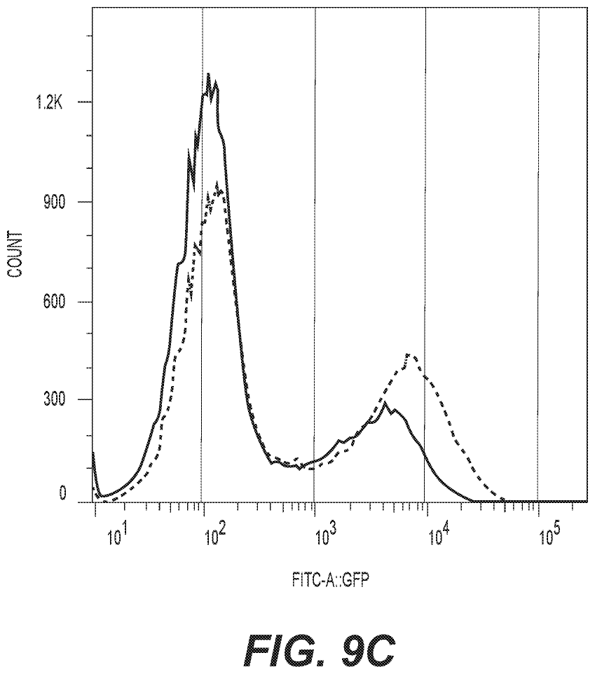

The expression of a detectable marker was observed in Ad biosensors 1 and 4 under bioreactor conditions, indicating that the biosensors were both infectious and functional. The expression level of the detectable marker by Ad biosensors 1 and 4, even though they had a leaky stop codon, was high enough for direct detection by FACS or a similar method. FACS analysis of the cell samples collected at 48 and 72 hours are shown in FIG. 9. The cells infected by control ChAd155-RSV virus exhibited a single peak of background autofluorescence when harvested at either 48 hours (solid lines) or 72 hours (dotted lines) (FIG. 9A).

As shown in FIG. 9B, Ad biosensor 4 infected cells exhibited a large EGFP signal peak corresponding to pIX fused to GFP at 48 hr (solid line) and a smaller peak at 72 hr (dotted line), consistent with the expression of a viral protein expressed at the intermediate stage of viral protein synthesis. After 48 hours of infection, up to 90% of the living cells were infected by Ad biosensor 4. The cell infection process, starting from receptor recognition, then E1 complementation and proceeding to early and intermediate gene expression occurred as expected, based on the control vector.

As expected with biosensor 1, the expression of the fiber was delayed compared to pIX. As shown in FIG. 9C the percentage of Ad biosensor 1 infected cells increased from 23% at 48 hours (solid line) to 39% at 72 hours (dotted line). This shows that the expression of EGFP fused to the late-expressed fiber protein was delayed compared to the expression of the intermediate-expressed pIX fused to EGFP. As expected, infection was not complete after 48 hours. Thus, biosensors of the invention identified the infection step reached by the replicating virus.

The phenotypes of fresh host cells and those frozen at 48 hr and 72 hr, which produced Ad biosensors 1 and 4 were analyzed by comparing the numbers of live, apoptotic and dead cells and also by comparing the number of cells displaying the detectable marker. As the viruses infected the cells, they naturally became apoptotic and died. The results of the fresh and frozen cells were comparable, demonstrating that the frozen cells were well preserved.

The phenotypes of the frozen samples harvested at 0, 6 h, 24 h, 30 h, 48 h, 54 h and 72 h after infection were then analyzed and the kinetics of expression characterized. EGFP expression was observed in cells infected by both Ad biosensor 1 and Ad biosensor 4 under bioreactor conditions, and the expression of Ad biosensor 1 (late expressed fiber) was delayed compared with the expression of Ad biosensor 4 (intermediate expressed pIX). Ad biosensor 4 was expressed mostly in non-apoptotic cells while Ad biosensor 1 was expressed mostly in the apoptotic cells that were observed later in the infection cycle.

As expected, the EGFP intensity differed between Ad biosensors 1 and 4 because the fiber and pIX were expressed by different promoters and the virus comprised more copies of EGFP on the larger fibers than on the smaller pIX proteins. The structural fiber protein was expressed from a strong late promoter and, at that expression step, viral mRNAs have been exported from the nucleus and the recognition of the mRNAs by the ribosomes is favored over the expression of cytoplasmic RNA messengers.

Up to 90% of the living cells were infected by biosensor 4 after 48 hr of infection and up to 50% of the living/apoptotic cells were infected by biosensor 1 after 48 hr, as shown by EGFP expression experiments. This demonstrates that the detectable marker was not toxic and that cell infection and viral replication proceeded according to the known kinetics of adenoviral infection and replication. Thus, most or all of the cells in the bioreactor were permissive for adenovirus infection and replication.

1. A recombinant nucleic acid encoding a fusion protein comprising a detectable marker and one or more viral proteins, wherein the detectable marker and the one or more viral proteins are expressed from the same precursor messenger RNA and wherein the nucleic acid encoding the one or more viral proteins comprises a leaky stop codon.

2. A virus comprising a recombinant nucleic acid encoding a fusion protein comprising a detectable marker and one or more viral proteins, wherein the detectable marker and the one or more viral proteins are expressed from the same precursor messenger RNA and wherein the nucleic acid encoding the one or more viral proteins comprises a leaky stop codon.

3. The recombinant nucleic acid of claim 1, wherein the nucleic acid further comprises an expression cassette comprising a transgene and regulatory elements necessary for the expression of the transgene in a host cell.

4. The recombinant nucleic acid of claim 1, wherein the detectable marker is selected from an affinity tag, an epitope tag and a fluorescent tag.

5.-6. (canceled)

7. The virus of claim 2, wherein the virus is an adenovirus, and wherein the adenovirus is a human or a simian adenovirus.

8.-9. (canceled)

10. The simian adenovirus of claim 7, wherein the simian adenovirus is selected from a bonobo, chimpanzee, gorilla, orangutan of rhesus monkey simian adenovirus.

11. The chimpanzee adenovirus of claim 10, wherein the chimpanzee adenovirus is selected from AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155 (WO 2016/198621), ChAd15, SadV41, ChAd157, ChAdOx1, ChAdOx2, sAd4287, sAd4310A, sAd4312, SAdV31 or SAdV-A1337.

12. The virus of claim 2, wherein the virus is replication competent.

13. The virus of claim 2, wherein the virus is replication defective.

14. The replication competent virus of claim 12, wherein the replication competent virus is an adenovirus.

15. The replication defective virus of claim 13, wherein the replication defective virus is an adenovirus, wherein the replication defective adenovirus lacks at least one gene of a genomic region selected from the group consisting of E1A, E1B, E2A, E2B, E3 and E4.

16.-25. (canceled)

26. The transgene of claim 3, wherein the transgene comprises one or more antigens, wherein the one or more antigens are prophylactic or therapeutic.

27. (canceled)

28. A vaccine comprising the recombinant nucleic acid of claim 1 and a transgene comprising one or more antigens, wherein the one or more antigens are prophylactic or therapeutic.

29.-30. (canceled)

31. The recombinant nucleic acid of claim 1, wherein the leaky stop codon replaces the corresponding natural stop codon.

32. The recombinant nucleic acid of claim 1, wherein the leaky stop codon is located at the junction between the viral protein and the detectable marker.

33. The recombinant nucleic acid of claim 1, wherein the leaky stop codon has the nucleic acid sequence TGAC.

34. The recombinant nucleic acid of claim 1, wherein the proportion of the viral proteins that fuse to the detectable marker is quantifiable and does not cause disruption to normal viral behavior, wherein the proportion is from about 0.1% to about 10%.

35. (canceled)

36. The recombinant nucleic acid of claim 1, wherein the nucleic acid is expressed from a host cell.

37-41. (canceled)

42. The adenovirus of claim 7, wherein the adenovirus comprises SEQ ID NO: 1.

43. The adenovirus of claim 7, wherein the adenovirus encodes one of the followings: SEQ ID NO: 2, SEQ ID NO: 3, SEQ ID NO: 8, SEQ ID NO 9, or SEQ ID NO: 10.

44.-52. (canceled)


SEQUENCE LISTING (17 sequences)

SEQ ID NO: 1 - DNA, 1875 bp, Artificial Sequence
  Description of Artificial Sequence Synthetic polynucleotide
tttaaagaattcagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaatgactaggcgggggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaataaaaagcatgacgctgttgatttgattcaatgtgtttctgttttattttcaagcagaattctttaaa

SEQ ID NO: 2 - protein, 578 aa, Artificial Sequence
  Description of Artificial Sequence Synthetic polypeptide
MKRTKTSDESFNPVYPYDTESGPPSVPFLTPPFVSPDGFQESPPGVLSLNLAEPLVTSHGMLALKMGSGLSLDDAGNLTSQDITTASPPLKKTKTNLSLETSSPLTVSTSGALTVAAAAPLAVAGTSLTMQSEAPLTVQDAKLTLATKGPLTVSEGKLALQTSAPLTAADSSTLTVSATPPLSTSNGSLGIDMQAPIYTTNGKLGLNFGAPLHVVDSLNALTVVTGQGLTINGTALQTRVSGALNYDTSGNLELRAAGGMRVDANGQLILDVAYPFDAQNNLSLRLGQGPLFVNSAHNLDVNYNRGLYLFTSGNTKKLEVNIKTAKGLIYDDTAIAINAGDGLQFDSGSDTNPLKTKLGLGLDYDSSRAIIAKLGTGLSFDNTGAITVGNKNDDKLTLWTTPDPSPNCRIYSEKDAKFTLVLTKCGSQVLASVSVLSVKGSLAPISGTVTSAQIVLRFDENGVLLSNSSLDPQYWNYRKGDLTEGTAYTNAVGFMPNLTAYPKTQSQTAKSNIVSQVYLNGDKSKPMTLTITLNGTNETGDATVSTYSMSFSWNWNGSNYINETFQTNSFTFSYIAQE

SEQ ID NO: 3 - protein, 820 aa, Artificial Sequence
  Description of Artificial Sequence Synthetic polypeptide
MKRTKTSDESFNPVYPYDTESGPPSVPFLTPPFVSPDGFQESPPGVLSLNLAEPLVTSHGMLALKMGSGLSLDDAGNLTSQDITTASPPLKKTKTNLSLETSSPLTVSTSGALTVAAAAPLAVAGTSLTMQSEAPLTVQDAKLTLATKGPLTVSEGKLALQTSAPLTAADSSTLTVSATPPLSTSNGSLGIDMQAPIYTTNGKLGLNFGAPLHVVDSLNALTVVTGQGLTINGTALQTRVSGALNYDTSGNLELRAAGGMRVDANGQLILDVAYPFDAQNNLSLRLGQGPLFVNSAHNLDVNYNRGLYLFTSGNTKKLEVNIKTAKGLIYDDTAIAINAGDGLQFDSGSDTNPLKTKLGLGLDYDSSRAIIAKLGTGLSFDNTGAITVGNKNDDKLTLWTTPDPSPNCRIYSEKDAKFTLVLTKCGSQVLASVSVLSVKGSLAPISGTVTSAQIVLRFDENGVLLSNSSLDPQYWNYRKGDLTEGTAYTNAVGFMPNLTAYPKTQSQTAKSNIVSQVYLNGDKSKPMTLTITLNGTNETGDATVSTYSMSFSWNWNGSNYINETFQTNSFTFSYIAQELGGGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 4 - DNA, 2002 bp, Artificial Sequence
  Description of Artificial Sequence Synthetic polynucleotide
tttaaagaattcggaatgtcagattcttgctcctgtccctccgcacccactatcttcatgttgttgcagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaataaaaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcggggaattctttaaa

SEQ ID NO: 5 - protein, 578 aa, Artificial Sequence; identical to SEQ ID NO: 2
  Description of Artificial Sequence Synthetic polypeptide

SEQ ID NO: 6 - DNA, 1522 bp, Artificial Sequence
  Description of Artificial Sequence Synthetic polynucleotide
tttaaagaattcgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgttgattacaaggatgacgacgataaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagataaataaaaccagactctgtttggattaaagaaaagtagcaagtgcattgctctctttagaattctttaaa

SEQ ID NO: 7 - protein, 401 aa, Artificial Sequence
  Description of Artificial Sequence Synthetic polypeptide
MSGSSSSSSSSSALDGSIVSPYLTTRMPHWAGVRQNVMGSSIDGRPVLPANSATLTYATVAGTPLDATAAAAATAAASAVRSLATDFAFLGPLATGATSRAAAAAVRDDKLTALLAQLDALTRELGDLSQQVMALRQQVSSLQAGGNASPTNAVDYKDDDDKMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - DNA, 1530 bp, Artificial Sequence
  Description of Artificial Sequence Synthetic polynucleotide
tttaaagaattccgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttgactagattacaaggatgacgacgataaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagataaataaaaccagactctgtttggattaaagaaaagtagcaagtgcattgctctctttatttcatgaattctttaaa

SEQ ID NO: 9 - protein, 154 aa, Artificial Sequence
  Description of Artificial Sequence Synthetic polypeptide
MSGSSSSSSSSSALDGSIVSPYLTTRMPHWAGVRQNVMGSSIDGRPVLPANSATLTYATVAGTPLDATAAAAATAAASAVRSLATDFAFLGPLATGATSRAAAAAVRDDKLTALLAQLDALTRELGDLSQQVMALRQQVSSLQAGGNASPTNAV

SEQ ID NO: 10 - protein, 402 aa, Artificial Sequence
  Description of Artificial Sequence Synthetic polypeptide
MSGSSSSSSSSSALDGSIVSPYLTTRMPHWAGVRQNVMGSSIDGRPVLPANSATLTYATVAGTPLDATAAAAATAAASAVRSLATDFAFLGPLATGATSRAAAAAVRDDKLTALLAQLDALTRELGDLSQQVMALRQQVSSLQAGGNASPTNAVLDYKDDDDKMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - DNA, 12 bp, Artificial Sequence
  Description of Artificial Sequence Synthetic oligonucleotide
ctaggcggggga

SEQ ID NO: 12 - protein, 4 aa, Artificial Sequence
  Description of Artificial Sequence Synthetic peptide
LGGG

SEQ ID NO: 13 - DNA, 24 bp, Artificial Sequence
  Description of Artificial Sequence Synthetic oligonucleotide
gattacaaggatgacgacgataaa

SEQ ID NO: 14 - protein, 8 aa, Artificial Sequence
  Description of Artificial Sequence Synthetic peptide
DYKDDDDK

SEQ ID NO: 15 - DNA, 27 bp, Artificial Sequence
  Description of Artificial Sequence Synthetic oligonucleotide
tagattacaaggatgacgacgataaaa

SEQ ID NO: 16 - protein, 9 aa, Artificial Sequence
  Description of Artificial Sequence Synthetic peptide
LDYKDDDDK

SEQ ID NO: 17 - protein, 8 aa, Artificial Sequence
  Description of Artificial Sequence Synthetic peptide
ITRMTTIK